(19) United States
(12) Patent Application Publication
(10) Pub. No.: US 2024/0239983 A1
(43) Pub. Date: Jul. 18, 2024

(54) METHOD FOR EXTRACTION AND TRANSFORMATION BY TRANSESTERIFICATION OF PHTHALATES CONTAINED IN PVC PLASTICS MATERIALS

(71) Applicant: IFP Energies Nouvelles, Rueil-Malmaison (FR)

(72) Inventors: Nicolas CADRAN, Rueil-Malmaison Cedex (FR); Alexandra CHAUMONNOT, Rueil-Malmaison Cedex (FR); Vincent COUPARD, Rueil-Malmaison Cedex (FR); Damien DELCROIX, Rueil-Malmaison Cedex (FR); Marc JACQUIN, Rueil-Malmaison Cedex (FR); David PASQUIER, Rueil-Malmaison Cedex (FR)

(73) Assignee: IFP Energies Nouvelles, Rueil-Malmaison (FR)

(21) Appl. No.: 18/561,764

(22) PCT Filed: May 5, 2022
(86) PCT No.: PCT/EP2022/062082

§ 371 Date: Nov. 17, 2023

(30) Foreign Application Priority Data
May 20, 2021 (FR) 2105300

(51) Int. Cl. C08J 11/02; B29B 17/02; B29B 17/04; B29K 27/06; B29K 105/00; C07C 67/02; C07C 67/58; C08J 11/04

(52) U.S. Cl. CPC C08J 11/02; B29B 17/0412; C07C 67/02; C07C 67/58; C08J 11/04; B29B 2017/0293; B29K 2027/06; B29K 2105/0038; C08J 2327/06

(57) ABSTRACT

The present invention relates to a process for obtaining a dialkyl phthalate and a reusable target PVC plastic from a PVC feedstock containing at least one phthalate, including:
a) solid-liquid extraction of PVC feedstock in the form of particles by placing the particles in contact with a solvent to produce a liquid phase enriched in the phthalate and a solid phase including PVC plastic depleted in the phthalate;
b) transformation of the phthalate of the liquid phase into dialkyl phthalate by transesterification using the solvent;
c) a solid-liquid separation between the solid phase and the liquid phase to produce at least one solid stream including the PVC plastic depleted in the phthalate so as to obtain the target PVC plastic;
d) a liquid-liquid separation of the liquid phase, to produce at least a first liquid effluent including the dialkyl phthalate and a second liquid effluent comprising said solvent.

### TECHNICAL FIELD

The invention relates to the field of the recycling of plastics based on polyvinyl chlorides (PVC), in particular to a process for extracting and transforming phthalates, which are plasticizers included in the composition of PVC, by transesterification. More precisely, the invention relates to a process for recovering a dialkyl phthalate (DAP) and a reusable target PVC plastic from a PVC feedstock containing at least one phthalate.

### PRIOR ART

By definition, a plastic is a mixture consisting of a base polymeric material and numerous additives, the assembly being able to be molded or fashioned (generally at elevated temperature and/or under pressure), so as to obtain a semifinished product or an object. A commonly accepted practice is to name said plastic by the name of the polymer of which it is made. Thus, the plastic polyvinyl chloride (PVC) in fact corresponds to the combination of the polymer PVC, referred to in the rest of the description as “PVC resin”, with various additives chosen as a function of the functionalities required for said plastic. Said additives may be organic molecules or macromolecules or alternatively inorganic (nano)particles and are used as a function of the properties that they afford to the PVC resin: heat resistance, light resistance or resistance to a mechanical stress (stabilizers), flexibility (plasticizers), processability (lubricants), coloring (dyes/pigments), etc.

Several methods for recycling PVC plastics exist: “conventional” methods by simple mechanical recycling of the plastics, methods involving modifications of their composition, or even chemical transformations of the compounds of which they are made.

Since the middle of the 20th century, the recycling of PVC plastic involving a chemical action has been the subject of numerous studies directed, in a first step, toward dissolving the PVC resin with a variable proportion of additives and then, in a second step, toward recovering said resin using various chemical processes (precipitation, evaporation, etc.) in the presence of all or some of the soluble additives. For example, patents EP0945481, EP1268628 and EP2276801 are directed, respectively, toward recycling various PVC-based objects (flexible or rigid pipes, window frames, cables, etc.) and specifically fiber-reinforced PVC-based objects (tarpaulins, floor coverings, etc.) according to a process involving a first step of dissolving the PVC resin and the soluble additives in an organic solvent, followed by a second step of precipitation with water vapor enabling the recovery of the resin and of the majority of the additives.

However, it is not always desirable to keep said additives in the PVC thus recovered to be recycled. For example, the changes over time in the regulations concerning them is a determining factor. Thus, certain plasticizers belonging to the phthalate family, which were notably widely used for formulating “flexible” PVCs about 40 years ago, gradually became subject to authorization in Europe on the basis of the REACH regulation, which, since the end of 2006, is directed toward establishing the safety of the manufacture and use of chemical substances in the European industry and, finally, were gradually excluded from the additives permitted for use. This is notably the case for the following nonexhaustive list of phthalates: dibutyl phthalate (DBP), dioctyl phthalate or diethyl hexyl phthalate (DOP or DEHP), benzyl butyl phthalate (BBP), diisobutyl phthalate (DIBP), dipentyl phthalate (DPP), diisopentyl phthalate, n-pentyl isopentyl phthalate, dihexyl phthalate, etc.

These new regulations are now leading toward the banning of the presence of such compounds in recycled raw materials (RRM). Taking into account the often very long service life of PVC-based objects (several decades), PVC-based objects formulated prior to the end of 2006 and now at the end of their service life cannot be recycled via regeneration methods leading to the maintenance of these banned additives, whether said methods are conventional, such as mechanical recycling processes, or not, for instance the dissolution/precipitation process examples mentioned above.

Moreover, the phthalate plasticizers currently used in Europe (REACH-compatible phthalates) and in the rest of the world represent high value-added additives which are not upgraded when they are kept in the PVC recycled raw material. The reason for this is that they are expensive products, present in appreciable proportions in the initial PVC formulations (several tens of percent), and cannot directly give the PVC RRM the adhoc flexibility properties. Supplying “fresh” plasticizers in appreciable amount is then essential for the reusability of the recycled PVC material.

The extraction of additives of phthalate type from PVC-based objects for removal or upgrading is thus a major challenge for optimized recyclability of PVC.

Several processes involving a step of dissolving the PVC resin have been adapted to enable this extraction. For example, patents EP1311599 and JP2007191586 both propose a first step of dissolving the PVC resin and of at least the phthalate-type additives with a first organic solvent, followed by a second step of liquid-liquid extraction of the phthalates from the solution obtained previously via the use of a second organic solvent different from the first organic solvent. Patent JP2007092035 discloses another possible example of implementation with dissolution of the PVC resin and of at least the phthalate-type additives via the use of a solvent under supercritical conditions and recovery of said phthalates in this same solvent after “rupture” of said supercritical conditions.

The removal or upgrading of phthalate-type additives from a PVC plastic may also be performed without proceeding via a preliminary step of dissolving said plastic, notably via direct extraction of said phthalates from the solid polymer matrix with a suitable organic solvent, as is fully indexed in the publication from Ogduler et al., 2020, “Challenge and opportunities of solvent-based additive extraction methods for plastic recycling”, Waste Management, 104, 148-182. The challenge then lies in optimizing the extraction conditions (nature of the solvent, contact time, temperature, pressure, etc.) to achieve the best possible yields of extracted phthalates. Although this methodology for the removal of phthalates from PVC plastics is frequently used, notably for detecting and analytically quantifying these specific additives in said plastics, to the Applicant's knowledge, no process for regenerating PVC-based objects involves this technique.

Although critical for ensuring efficient recycling of PVC plastics and for obtaining a reusable recycled PVC, the extraction of phthalate-type plasticizers is insufficient to ensure the economic viability of a process for regenerating PVC-based objects. The main reason frequently put forward is the difficulty in finding an economically viable balance between the cost of the individual operations performed in said regeneration process and the resale cost (equivalent to the added value) of the products obtained. Said products consist of the phthalate-free PVC-based recycled material, which is naturally upgradable, and of said extracted phthalates which are, themselves, sparingly upgradable. Specifically, any regeneration process involving a step of extracting the phthalates from PVC-based objects will lead to the recovery of a mixture of phthalates which may comprise phthalates that are not “REACH-compatible”. The upgrading of said non-REACH-compatible phthalates is, of course, excluded and said phthalates will need to be treated as specific waste giving rise to additional costs. The upgrading of the REACH-compatible phthalates, which is advantageous per se, is in point of fact difficult since it involves technically complex and expensive separation/purification steps.

### SUMMARY OF THE INVENTION

The present invention is aimed at overcoming, at least partly, the problems of the prior art and is particularly directed toward providing a process for regenerating PVC-based objects allowing the treatment of any type of PVC feedstock containing phthalates and their transformation into two products of interest that can be upgraded: a specific dialkyl phthalate and a recyclable PVC plastic free of phthalates, notably of undesirable phthalates, typically those that are subject to authorization by the European REACH regulation. Another aim of the present invention is to limit, during the recycling of PVC containing phthalates, the number of individual steps conventionally associated with the phthalate separation/purification operations, thus making it possible to limit the process costs.

Thus, to achieve at least one of the abovementioned objectives, among others, the present invention proposes, according to a first aspect, a process for recovering a dialkyl phthalate and a reusable target PVC plastic from a PVC feedstock containing at least one phthalate, including the following steps:
a) solid-liquid extraction of said PVC feedstock in the form of particles by placing said particles of the PVC feedstock in contact with a solvent including at least one chemical molecule of ester, ether, ketal or acetal type, of empirical formula (CₙH₂ₙ₊₁O)ₘZ, n and m being positive integers with n<4 or n>8, m greater than or equal to 1 and less than or equal to 3, and Z being a group chosen from the list consisting of the following elements: R, COOR, CO, CR, CNRR′, PO, P, SO, SO2, COR, and HCO, and with R and R′ chosen independently from a linear, branched or cyclic alkyl group, or an aryl group, to produce a liquid phase enriched in said phthalate and a solid phase including PVC plastic depleted in said phthalate;
b) chemical transformation of said phthalate of said liquid phase into dialkyl phthalate of formula C₆H₄(COOCₙH₂ₙ₊₁)₂ by transesterification using said chemical molecule of ester, ether, ketal or acetal type of empirical formula (CₙH₂ₙ₊₁O)ₘZ to enrich said liquid phase in said dialkyl phthalate;
c) a solid-liquid separation between said solid phase and said liquid phase to produce at least one solid stream including the PVC plastic depleted in said phthalate so as to recover said target PVC plastic;
d) a liquid-liquid separation of said liquid phase, to produce at least a first liquid effluent including said dialkyl phthalate and a second liquid effluent comprising at least said solvent.

One advantage of the present invention lies in the ability of the process, by means of a chemical transesterification reaction, to transform a mixture of phthalates initially trapped in polymeric matrices of various objects based on PVC plastic, irrespective of the composition of said mixture (i.e. irrespective of the nature and origin of the various phthalates) and despite the possible presence of numerous other additives, into a single REACH-compatible and upgradable phthalate product of DAP type. The production of the single specific DAP product from the mixture of phthalates also makes it possible to limit the number of individual steps associated with the separation/purification operations and thus to limit the costs.

According to a first variant, steps a) and b) are performed within the same individual operation.

According to a second variant alternative to the first variant, steps a) and b) form the subject of two distinct individual operations, step a) producing a stream including the liquid phase and the solid phase.

According to this second variant, step c) may be performed between steps a) and b), the stream including the liquid phase and said solid phase obtained from step a) possibly being sent into the solid-liquid separation step c) to produce the stream including the PVC plastic depleted in said phthalate and a first liquid stream including the liquid phase sent into step b).

According to one or more embodiments, the process also comprises an additional step f₁) of chemical transformation by transesterification of said phthalate which is unconverted and/or partially converted in step b), into dialkyl phthalate of formula C₆H₄(COOCₙH₂ₙ₊₁)₂ by means of said solvent, said step f₁) being performed between steps c) and d) by sending said liquid phase obtained on conclusion of all the steps a), b) and c) into a first additional transesterification reactor to produce a second liquid stream enriched in said dialkyl phthalate of formula C₆H₄(COOCₙH₂ₙ₊₁)₂, said second liquid stream being sent into step d).

According to one or more embodiments, said solvent is supplied and/or at least a portion of said second liquid effluent comprising at least said solvent obtained from step d) is recycled into the first additional transesterification reactor.

According to one or more embodiments, in step d), said first effluent consists essentially of said dialkyl phthalate.

According to one or more embodiments, the liquid-liquid separation step d) also produces a third effluent including byproducts of ester, ether, ketal or acetal type obtained during step b) and optionally a fourth effluent including said phthalate that is partially converted and/or unconverted in step b) and optionally other soluble impurities, said first liquid effluent consisting essentially of said dialkyl phthalate and said second liquid effluent consisting essentially of said solvent.

According to one or more embodiments, the liquid-liquid separation step d) also produces a third effluent including byproducts of ester, ether, ketal or acetal type obtained during step b), said first liquid effluent comprising said dialkyl phthalate, phthalate that is partially converted and/or unconverted in step b) and optionally soluble impurities, said second liquid effluent consisting essentially of said solvent, and the process also comprises:
e) purification of said first liquid effluent to produce a liquid product consisting essentially of said dialkyl phthalate, and a liquid residue comprising said phthalate that is partially converted and/or unconverted in step b) and optionally said soluble impurities.

According to one or more embodiments, the process also comprises an additional step f₂) of chemical transformation by transesterification of said phthalate which is unconverted and/or partially converted in step b), into dialkyl phthalate of formula C₆H₄(COOCₙH₂ₙ₊₁)₂ by means of said solvent, said step f₂) being performed after step e) by sending said liquid residue into a second additional transesterification reactor to produce a third liquid stream enriched in said dialkyl phthalate of formula C₆H₄(COOCₙH₂ₙ₊₁)₂, said third liquid stream being returned into step d).

According to one or more embodiments, said solvent is supplied and/or at least a portion of said second liquid effluent comprising at least said solvent obtained from step d) is recycled into the second additional transesterification reactor.

According to one or more embodiments, the process also comprises the recycling of at least a portion of said liquid residue into step b) and/or an additional step f₁) of chemical transformation by transesterification of said phthalate which is unconverted and/or partially converted in step b), into dialkyl phthalate of formula C₆H₄(COOCₙH₂ₙ₊₁)₂ by means of said solvent, said step f₁) being performed between steps c) and d) by sending said liquid phase obtained on conclusion of all the steps a), b) and c) into a first additional transesterification reactor to produce a second liquid stream enriched in said dialkyl phthalate of formula C₆H₄(COOCₙH₂ₙ₊₁)₂, said second liquid stream being sent into step d).

According to one or more embodiments, the second liquid effluent comprising at least said solvent obtained from step d) is at least partly recycled into step a) and/or step b).

According to one or more embodiments, the solid stream including the PVC plastic depleted in phthalates is at least partly recycled into step a).

According to one or more embodiments, the chemical molecule of said solvent is an ester bearing one or more alkoxy groups of formula (CₙH₂ₙ₊₁O)ₘ, with n<4 or n>8 and m greater than or equal to 1 and less than or equal to 3, said ester being preferably chosen from the list consisting of the carboxylic esters of formula (CₙH₂ₙ₊₁O)COR, the carbonate esters of formula (CₙH₂ₙ₊₁O)₂CO, the orthoesters of formula (CₙH₂ₙ₊₁O)₃CR, the imino esters of formula (CₙH₂ₙ₊₁O)CNRR′, the phosphite esters of formula (CₙH₂ₙ₊₁O)₃P, the phosphate esters of formula (CₙH₂ₙ₊₁O)₃PO, the sulfite esters of formula (CₙH₂ₙ₊₁O)₂SO, the sulfate esters of formula (CₙH₂ₙ₊₁O)₂SO₂, and mixtures thereof, with the proviso that the esters involved in said mixtures contain alkoxy groups CₙH₂ₙ₊₁O with a value of n that is strictly identical, and more preferentially said chemical molecule of said solvent (9) is a carboxylic ester of formula (CₙH₂ₙ₊₁O)COR with n<4 or n>8, preferably chosen from the list consisting of methyl acetate, ethyl acetate, propyl acetate, isopropyl acetate, nonyl acetate, which may be linear or branched, decyl acetate, which may be linear or branched, methyl propanoate, ethyl propanoate, propyl propanoate, isopropyl propanoate, nonyl propanoate, which may be linear or branched, and decyl propanoate, which may be linear or branched, and is preferably methyl acetate or methyl propanoate.

According to one or more embodiments, the chemical molecule of said solvent is an ether of formula (CₙH₂ₙ₊₁O)R, with n<4 or n>8, preferably chosen from the list consisting of dimethyl ether, diethyl ether, dipropyl ether, diisopropyl ether, dinonyl ether, which may be linear or branched, didecyl ether, which may be linear or branched, and more preferentially is dimethyl ether or diethyl ether.

According to one or more embodiments, the chemical molecule of said solvent is a ketal or acetal of formula, respectively, (CₙH₂ₙ₊₁O)₂CRR′ or (CₙH₂ₙ₊₁O)₂CRH, with n<4 or n>8, preferably chosen from the list consisting of dimethylal, 2,2-dimethoxypropane, 2,2-dimethoxybutane, diethyl acetal, 2,2-diethoxypropane and 2,2-dipropoxypropane, and more preferentially is dimethylal, 2,2-dimethoxypropane or 2,2-dimethoxybutane.

According to one or more embodiments, the chemical molecule of said solvent is methyl propanoate and said dialkyl phthalate is dimethyl phthalate.

According to one or more embodiments, the chemical transformation performed by transesterification in step b), and optionally in step f₁) and/or f₂), is performed using a transesterification catalyst preferably chosen from the list consisting of mineral or organic basic or acidic Brønsted homogeneous catalysts, or Lewis acids, and heterogeneous catalysts formed by alkaline-earth metal oxides, or alkali metal and/or alkaline-earth metal carbonates or hydrogen carbonates, or alkali metals supported on aluminas or zeolites, or zinc oxides and mixtures thereof with other oxides, or ion-exchange resins.

According to one or more embodiments, said at least one phthalate of said PVC feedstock is a phthalate of empirical formula C₆H₄(COOR₁)(COOR₂) in which the ester groups are in the ortho position of the benzene nucleus, R₁ or R₂ being chosen independently from one of the elements of the group consisting of a linear or branched or cyclic alkyl chain, a linear or branched alkoxyalkyl chain, or an aryl or alkylaryl chain, R₁ and/or R₂ preferably comprising between 1 and 20 carbon atoms, or even between 1 and 15 carbon atoms.

According to one or more embodiments, the target PVC plastic is substantially free of said phthalate, and preferably comprises less than 0.1% by mass in total of phthalates chosen from the list consisting of dibutyl phthalate, dioctyl phthalate or diethylhexyl phthalate, benzyl butyl phthalate, dibutyl phthalate, diisobutyl phthalate, dipentyl phthalate, diisopentyl phthalate, n-pentyl isopentyl phthalate, dihexyl phthalate, bis(2-methoxyethyl) phthalate, and mixtures thereof.

According to one or more embodiments, step b), and optionally steps f₁) and/or f₂), are performed at a temperature of between room temperature and 200° C., preferably between 40° C. and 180° C., at a pressure of between atmospheric pressure and 11.0 MPa, preferably between atmospheric pressure and 5.0 MPa, and for a time of between 1 minute and 10 hours, preferably between 10 minutes and 4 hours.

According to one or more embodiments, step a) and/or step b), and optionally steps f₁) and/or f₂), are performed such that the mole ratio between the amount of said solvent (9) and the amount of said phthalate to be extracted or to be transformed is between 2 and 250, preferably between 4 and 90.

According to a second aspect, the present invention relates to a process for recycling a PVC-based object containing at least one phthalate, including:
the conditioning of said PVC-based object comprising at least milling or shredding of said PVC-based object to form a PVC feedstock in the form of particles;
the recovery of a dialkyl phthalate and of a reusable target PVC plastic from said PVC feedstock in the form of particles according to the invention.

According to a third aspect, the present invention relates to a process for manufacturing a flexible PVC-based object including a recycled PVC plastic and/or a dialkyl phthalate which are obtained via the process for recovering a dialkyl phthalate and a reusable target PVC plastic according to the invention.

Other subjects and advantages of the invention will become apparent on reading the description which follows of particular implementation examples of the invention, which are given as nonlimiting examples, the description being made with reference to the appended figures described below.

### LIST OF FIGURES

FIG. 1 is a scheme of the process according to one embodiment of the invention including steps a), b), c) and d).

FIG. 2 is a scheme of the process according to another embodiment including steps a), b), c) and d), with, in step d), a separation between the DAP, the solvent, byproducts obtained in step b) of ester, ether, ketal or acetal type, and the partially converted and/or unconverted phthalates optionally as a mixture with soluble impurities.

FIG. 3 is a scheme of the process according to the embodiments illustrated in FIG. 1 or in FIG. 2, including steps a), b), c) and d), and illustrating the implementation of other optional steps of transesterification (f₁) and of recycling of various streams.

FIG. 4 is a scheme of the process according to another embodiment of the invention including steps a), b), c) and d) and also a step e) of purification of a first effluent obtained in step d) comprising DAP.

FIG. 5 is a scheme of the process according to the embodiment illustrated in FIG. 4, and illustrating the implementation of other optional steps of transesterification (f₁; f₂) and of recycling of various streams.

FIG. 6 is a scheme of the process according to a preferred embodiment of the invention, including an implementation within the same individual operation of steps a) and b) (first variant of the process according to the invention), a step e) of purification of a first effluent obtained in step d) comprising DAP and an additional step of transesterification f₂) of the residue obtained from step e).

FIG. 7 is a scheme of the process according to another embodiment of the invention including steps a), b), c) and d), in which steps a) and b) form the subject of two distinct individual operations (second variant of the process according to the invention), and in which step c) is performed between steps a) and b).

FIG. 8 is a scheme of the process as illustrated in FIG. 7, according to a preferred embodiment including a step e) of purification of a first effluent obtained in step d) comprising DAP and an additional step of transesterification f₂) of the residue obtained from step e).

In the figures, the same references denote identical or analogous elements.

### DESCRIPTION OF THE EMBODIMENTS

### Terminology

Certain definitions are given below, although further details regarding the objects defined hereinbelow may be given later in the description.

The term “PVC-based object” means an object, generally a consumer object, which comprises, and preferably consists of, at least one PVC plastic.

The term “polyvinyl chloride plastic”, also known as PVC plastic or simply PVC, means the combination of a PVC polymer, also known as PVC resin, with various additives chosen as a function of the functionalities required for the PVC plastic, which are themselves chosen as a function of the intended applications.

Said PVC polymer is derived from the radical polymerization of vinyl chloride (VCM), which monomer is itself obtained from chlorine and ethylene. Depending on the implementation of said polymerization, four families of PVC resins may be used: 1) suspension PVC or S-PVC resins (suspension polymerization of VCM), 2) emulsion PVC or PVC “paste” resins (emulsion polymerization), 3) mass PVC or M-PVC resins (mass polymerization) and 4) superchlorinated PVC or C-PVC resins, obtained by superchlorination as a post-treatment on the preceding resins.

Said additives included in the composition of a PVC plastic may be organic molecules or macromolecules or alternatively inorganic (nano)particles and are used as a function of the properties that they afford to the PVC resin: heat resistance, light resistance or resistance to a mechanical stress (stabilizers), flexibility (plasticizers), processability (lubricants), coloring (dyes/pigments), etc.

The term “phthalates” means the group of chemical products formed by dicarboxylic esters of o-phthalic acid. They are composed of a benzene nucleus and of two carboxylic ester groups placed in the ortho position on the benzene nucleus. They may be described by means of the following formula:

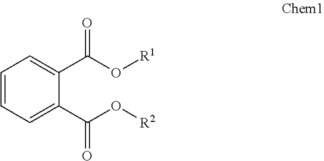

or alternatively by the empirical formula C₆H₄(COOR₁)(COOR₂), in which R₁ and R₂ are independently chosen from one of the elements of the group consisting of a linear, branched or cyclic alkyl chain, a linear or branched alkoxyalkyl chain, or an aryl or alkylaryl chain. Said alkyl, alkoxyalkyl, aryl or alkylaryl chain may typically include between 1 and 20 carbon atoms, or may even include between 1 and 15 carbon atoms.

For example, R₁ and/or R₂ may be chosen from ethyl, n-butyl, isobutyl, n-pentyl, iso-pentyl, n-hexyl, n-octyl, n-nonyl, n-decyl, isodecyl, methoxyethyl and benzyl groups. Phthalates are commonly used as plasticizers for plastics and in particular as plasticizers for plastics of PVC type, notably to make them flexible.

In the present description, the term “transesterification” denotes the chemical reaction for transforming at least one carboxylic ester function —COOR₁ or —COOR₂ of a phthalate as defined above into a new carboxylic ester function —COO(CₙH₂ₙ₊₁), with n<4 or n>8, irrespective of the reagent used.

In the present description, the term “dialkyl phthalate” (DAP) denotes the product of empirical formula C₆H₄(COOCₙH₂ₙ₊₁)₂ resulting from the transesterification reaction between at least one plasticizer of phthalate type (and in particular of empirical formula C₆H₄(COOR₁)(COOR₂) as described above) present in PVC-based objects with any chemical molecule of ester, ether, ketal or acetal type of empirical formula (CₙH₂ₙ₊₁O)ₘZ, which may also be called the “reagent” or “solvent” in the rest of the description, n and m being positive integers such that n<4 or n>8 and m is greater than or equal to 1 and less than or equal to 3, Z being a group chosen from the list consisting of one of the following elements: R, COOR, CO, CR, CNRR′, PO, P, SO, SO2, COR, and HCO, with R and R′ chosen independently from a (linear, branched or cyclic) alkyl or aryl group, for example comprising between 1 and 20 carbon atoms, or even between 1 and 15 carbon atoms. Said chemical molecule is described in greater detail later in the description. Dimethyl phthalate is an example of DAP.

The term “byproducts of ester, ether, ketal or acetal type” (BP) means the byproducts of formula R₁OZ or R₂OZ resulting from the transesterification reaction between at least one plasticizer of phthalate type (and in particular of empirical formula C₆H₄(COOR₁)(COOR₂) as described above) present in PVC-based objects with the reagent (chemical molecule of ester, ether, ketal or acetal type of empirical formula (CₙH₂ₙ₊₁O)ₘZ as defined above). R₁ and R₂ are defined identically to R₁ and R₂ of the phthalate.

Z is defined identically to Z of the reagent.

The term “intermediate alkyl phthalate” (IAP) means the byproduct of empirical formula C₆H₄(COOR₁)(COOCₙH₂ₙ₊₁) or C₆H₄(COOR₂)(COOCₙH₂ₙ₊₁) resulting from the incomplete transesterification reaction between at least one plasticizer of phthalate type (and in particular of empirical formula C₆H₄(COOR₁)(COOR₂) as described above) present in PVC-based objects with the reagent (chemical molecule of ester, ether, ketal or acetal type of empirical formula (CₙH₂ₙ₊₁O)ₘZ as defined above). R₁ and R₂ are defined identically to R₁ and R₂ of the phthalate. Z is defined identically to Z of the reagent.

The term “reusable target PVC plastic” means a “phthalate-free PVC”, i.e. the solid comprising at least one PVC resin supplemented with at least one of the additives initially present in the PVC plastic of the PVC feedstock treated according to the invention, and from which the phthalates have been extracted and transformed in the form of at least one dialkyl phthalate according to the invention. The term “phthalate-free” in particular means that the solid PVC obtained as product of the process according to the invention contains, in total, less than 0.1% by weight of phthalates subject to authorization by the REACH regulation in Europe (appendix XIV of the regulation (EC) Number 1907/2006 of the European Parliament and of the Council of 18 Dec. 2006), in particular less than 0.1% by weight of phthalates, chosen from the list consisting of the following phthalates: dibutyl phthalate (DBP), dioctyl phthalate or diethylhexyl phthalate (DOP or DEHP), benzyl butyl phthalate (BBP), dibutyl phthalate (DBP), diisobutyl phthalate (DIBP), dipentyl phthalate (DPP), diisopentyl phthalate, n-pentyl isopentyl phthalate, dihexyl phthalate, bis(2-methoxyethyl) phthalate, alone or as a mixture.

In the present description, the term “greater than . . . ” is understood as strictly greater than, and is symbolized by the sign “>”, and the term “less than . . . ” is understood as strictly less than, and is symbolized by the sign “<”.

In the present description, the indices “n” and “m” in the cited chemical formulae are positive integers (i.e. strictly greater than zero). According to the invention, n is less than 4 or greater than 8, and preferably less than or equal to 20, or even less than or equal to 15. According to the invention, m is an integer greater than or equal to 1 and less than or equal to 3.

In the present description, the term “room temperature” (r.t.) means a temperature typically of 20° C.±5° C., and the term “atmospheric pressure” means a pressure of 0.101325 MPa.

In the present description, the term “comprise” is synonymous with (means the same as) “include” and “contain”, and is inclusive or open-ended and does not exclude other elements which are not specified. It is understood that the term “comprise” includes the exclusive and closed term “consist”.

In the present description, the expression “between . . . and . . . ” means that the limit values of the interval are included in the described range of values, unless otherwise mentioned.

In the present description, the various ranges of parameters for a given step, such as the pressure ranges and the temperature ranges, may be used alone or in combination. For example, in the present description, a range of preferred pressure values can be combined with a range of more preferred temperature values.

In the text hereinbelow, particular embodiments of the invention may be described. They may be implemented separately or combined together without limitation of combinations when this is technically feasible.

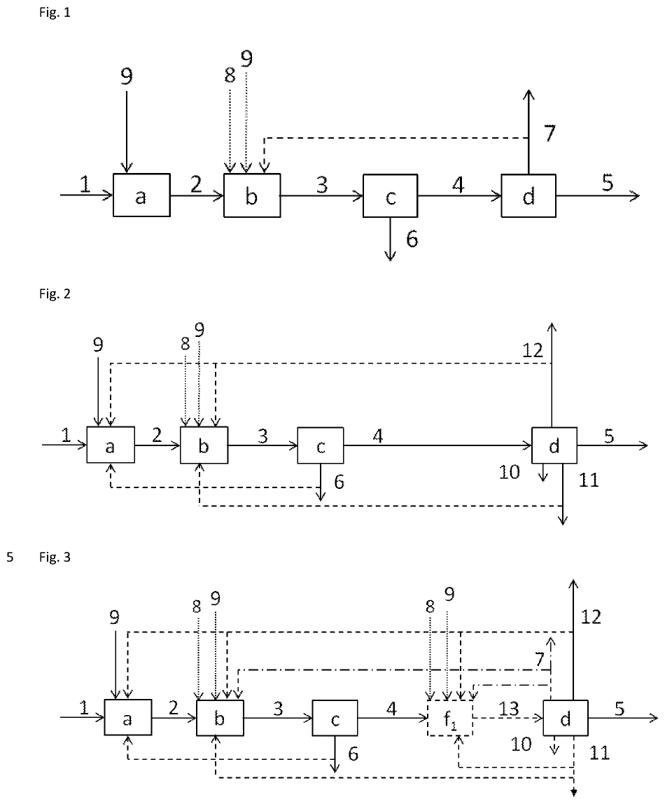

The below description of the process according to the invention refers to the schemes in FIGS. 1 to 8, illustrating various implementations of the process according to the invention.

In accordance with the invention, the process for recovering a DAP and a reusable target PVC plastic from a PVC feedstock containing at least one phthalate includes, and may consist of, the following steps:
a) solid-liquid extraction of said PVC feedstock in the form of particles 1 by placing said particles of the PVC feedstock in contact with a solvent 9 including at least one chemical molecule of ester, ether, ketal or acetal type, of empirical formula (CₙH₂ₙ₊₁O)ₘZ, n and m being positive integers with n<4 or n>8, m greater than or equal to 1 and less than or equal to 3, and Z being a group chosen from the list consisting of one of the following elements: R, COOR, CO, CR, CNRR′, PO, P, SO, SO2, COR, and HCO, and with R and R′ chosen independently from a linear, branched or cyclic alkyl group, or an aryl group, to produce a liquid phase enriched in said phthalate and a solid phase including PVC plastic depleted in said phthalate;
b) chemical transformation of said phthalate of said liquid phase into dialkyl phthalate of formula C₆H₄(COOCₙH₂ₙ₊₁)₂ by transesterification using said chemical molecule of ester, ether, ketal or acetal type of empirical formula (CₙH₂ₙ₊₁O)ₘZ to enrich said liquid phase in said dialkyl phthalate;
c) a solid-liquid separation between said solid phase and said liquid phase to produce at least one solid stream including the PVC plastic depleted in said phthalate 6 so as to recover said target PVC plastic;
d) a liquid-liquid separation of said liquid phase 4, to produce at least a first liquid effluent including said dialkyl phthalate (5 or 14) and a second liquid effluent comprising at least said solvent (7 or 12).

### Feedstock

The process according to the invention is fed with a feedstock known as “PVC feedstock” 1 comprising at least one PVC plastic, which necessarily comprises at least one phthalate as described in the present invention.

Said PVC plastic may include at least 0.1% by mass of phthalates, or even at least 1% by mass of phthalates or else at least 5% by mass of phthalates. In general, the PVC plastics advantageously comprise less than 60% by weight of phthalates, typically less than 30% by weight of phthalates.

Said PVC feedstock is advantageously a feedstock of PVC to be recycled of the “production scraps” type, i.e. waste resulting from the processes for producing the PVC polymer during its polymerization or from the PVC plastic during its formulation/forming or of the PVC-based object during its production, or of the “post-consumption waste” type, i.e. waste generated after consumption by the user of said PVC-based object.

In particular, the PVC feedstock to be recycled may be derived from any existing collection and sorting channels or networks for production scraps and/or post-consumption waste making it possible to isolate a stream based on at least one PVC plastic comprising at least one phthalate, notably the collection and sorting channels or networks specific to plastic waste.

Thus, the PVC feedstock, which is typically of “production scraps” type and/or of “post-consumption waste” type, generally comes from the main fields of application which use PVC plastic, such as, in a nonexhaustive manner, the building and construction sectors, packaging, motor vehicles, electrical and electronic equipment, sports, medical equipment, etc. Preferably, the PVC feedstock comes from the building and construction sector. More precisely, the PVC-based objects are generally used in these fields as various rigid profiles (windows, doors, awnings, roller blind casings), pipes and connections, and rigid bottles, plates and films, flexible films and sheets, flexible tubes and profiles, cables, floor coverings, coated fabrics, etc. Preferably, the PVC-based objects forming the PVC feedstock comprise at least one “flexible” PVC, i.e. PVC containing additives of plasticizer type, preferably of phthalate type, as is the case, for example, for the following PVC-based objects: flexible films and sheets, flexible tubes and profiles, cables, floor coverings, coated fabrics, etc.

Advantageously, the PVC feedstock comprises at least 50% by mass, preferably at least 70% by mass, preferably at least 90% by mass and even more preferably at least 95% by mass of PVC plastic comprising at least one phthalate.

Preferably, the PVC feedstock comprises “flexible” PVC, i.e. PVC containing additives of plasticizer type, preferably of phthalate type.

Even more preferably, the PVC feedstock predominantly or even exclusively comprises “flexible” PVC, i.e. PVC containing additives of plasticizer type, preferably of phthalate type.

The PVC feedstock treated in the process for recovering a DAP and a reusable target PVC plastic according to the invention is in the form of particles. Thus, if the PVC feedstock is in an initial form which is that specific to production scraps or post-consumption waste, notably, in the latter case, in the initial form of PVC-based objects, it may first undergo a conditioning step comprising at least milling or shredding to form a PVC feedstock in the form of particles. Depending on the channels and/or networks from which these production scraps and/or PVC-based objects at the end of their service life are derived, the PVC waste may be milled and/or washed and/or may undergo any other conditioning step as described hereinbelow, so as to form the PVC feedstock in the form of particles that are suitable for the process according to the invention. For example, the PVC feedstock may advantageously be in the form of milled and optionally washed material, the largest dimension of which is less than 20 cm, preferably less than 10 cm, preferably less than 1 cm and even more preferably less than 5 mm. The PVC feedstock may also advantageously be in micronized solid form, i.e. in the form of particles preferably having a mean size of less than 1 mm, for example between 10 micrometers (μm) and 800 micrometers (μm). The mean size advantageously corresponds to the mean diameter of the spheres in which said particles are circumscribed.

Thus, the term “PVC feedstock in the form of particles” means particles of PVC plastic typically having a mean size, as defined previously, of between 10 μm and 20 cm, for example particles of milled material type with a mean size of between 1 mm and 20 cm, preferably between 1 mm and 10 cm, more preferentially between 1 mm and 1 cm, even more preferentially between 1 mm and 5 mm, or particles derived from micronization (very fine milling to produce a powder) with a mean size of less than 1 mm, preferably between 10 μm and 800 μm.

Preferably, the PVC feedstock treated in the process according to the invention is in the form of particles of milled material type, preferably particles with a mean size of between 1 mm and 5 mm, or particles derived from micronization (very fine milling to produce a powder) with a mean size of less than 1 mm.

The PVC feedstock may also comprise “macroscopic” impurities, such as glass, metal, plastics other than PVC (for example PET, etc.), wood, paper, cardboard, mineral elements, etc. Advantageously, the PVC feedstock comprises not more than 50% by mass, preferably not more than 30% by mass, preferably not more than 10% by mass and even more preferably not more than 5% by mass of “macroscopic” impurities.

Advantageously, the PVC feedstock in the form of particles has a water content of less than or equal to 0.3% by mass and preferably less than or equal to 0.1% by mass.

The various steps of the process according to the invention leading to the DAP and to the reusable target PVC plastic are detailed in the paragraphs that follow.

### Optional Preliminary Step of Conditioning the PVC Feedstock

According to the invention, the process may comprise a preliminary step of conditioning the PVC feedstock (not shown in the figures) including at least one step of milling or shredding the PVC feedstock to form a PVC feedstock in the form of solid particles as defined above, which can be sent into the solid-liquid extraction step a). This preconditioning step may also comprise one or more steps mentioned in the following nonexhaustive list: milling by micronization, sorting, oversorting, washing, drying, etc. Depending on the nature of the PVC feedstock treated, the step or steps, and their possible frequencies and sequences, involved in the preconditioning step are notably chosen by a person skilled in the art so as to limit the amount of macroscopic impurities and to reduce the size of the solid elements of which the PVC feedstock is initially composed.

For example, the preconditioning step makes it possible to provide a PVC feedstock in the form of particles, for example washed milled material with a mean size of less than 5 mm, the macroscopic impurity content of which is preferably not more than 10% by mass and more preferably not more than 5% by mass. Said preconditioned PVC feedstock may also be in the form of micronized solid particles, i.e. in the form of particles with a mean size of less than 1 mm, for example between 10 μm and 800 μm.

The step of preconditioning of the PVC feedstock preferably comprises at least one step of drying the PVC feedstock which is already in the form of solid particles of ad hoc size and macroscopic impurity content, such that said PVC feedstock has a residual water content of not more than 0.3% by mass and preferably not more than 0.1% by mass.

### Step a) of Solid-Liquid Extraction of the Phthalates

The process according to the invention comprises a step a) of solid-liquid extraction of the phthalate(s) of the PVC feedstock in the form of particles 1 by placing said feedstock 1 in contact with a solvent 9 including, and preferably consisting of, the chemical molecule of ester, ether, ketal or acetal type of empirical formula (CₙH₂ₙ₊₁O)ₘZ, with n<4 or n>8, m greater than or equal to 1 and less than or equal to 3, and Z being a group chosen from the list consisting of one of the following elements: R, COOR, CO, CR, CNRR′, PO, P, SO, SO2, COR, and HCO, with R and R′ chosen independently from a (linear, branched or cyclic) alkyl or aryl group, for example comprising between 1 and 20 carbon atoms, or even between 1 and 15 carbon atoms, so as to obtain an effluent 2 comprising at least a liquid phase and a solid phase.

In particular, in the formula (CₙH₂ₙ+1O)ₘZ:
when m=1, Z is chosen among R, COOR, COR, CNRR′, HCO;
when m=2, Z is chosen among CO, SO, SO2;
when m=3, Z is chosen among PO, P, CR.

Said liquid phase is then enriched in said phthalate(s), and the solid phase includes PVC plastic depleted in said phthalate(s).

The specific choice of n for the chemical molecule of ester, ether, ketal or acetal type of the solvent (exclusion, for the choice of the alkyl chain CₙH₂ₙ₊₁, of C4, C5, C6, C7 and C8 chains) makes it possible, during step b), to transform, by transesterification by means of said chemical molecule of ester, ether, ketal or acetal type, said phthalates into at least one DAP as defined in the present description, which is not among the undesirable phthalates such as those that are subject to authorization by the REACH regulation discussed hereinabove.

According to one or more embodiments, said chemical molecule of ester, ether, ketal or acetal type has the empirical formula (CₙH₂ₙ₊₁O)ₘZ with n<4, preferably n=1 or n=2, and even more preferably n=1.

According to one or more embodiments, said chemical molecule of ester, ether, ketal or acetal type has the empirical formula (CₙH₂ₙ₊₁O)ₘZ with n>8 and n less than or equal to 20, or even n less than or equal to 15.

Preferably, n is an integer such that n<4 and more preferably n is equal to 1 or 2.

According to one or more embodiments, said chemical molecule is an ester bearing one or more alkoxy groups of formula (CₙH₂ₙ₊₁O)ₘ with n<4 or n>8 and m greater than or equal to 1 and less than or equal to 3.

Said ester is preferably chosen from the list consisting of: the carboxylic esters of formula (CₙH₂ₙ₊₁O)COR, the carbonate esters of formula (CₙH₂ₙ₊₁O)₂CO, the orthoesters of formula (CₙH₂ₙ₊₁O)₃CR, the imino esters of formula (CₙH₂ₙ₊₁O)CNRR′, the phosphite esters of formula (CₙH₂ₙ₊₁O)₃P, the phosphate esters of formula (CₙH₂ₙ₊₁O)₃PO, the sulfite esters of formula (CₙH₂ₙ₊₁O)₂SO, the sulfate esters of formula (CₙH₂ₙ₊₁O)₂SO₂, the formic acid esters (alkyl formates) of formula (CₙH₂ₙ₊₁O)HCO, for instance ethyl formate or methyl formate, and mixtures thereof, with the proviso that the esters involved in said mixtures contain alkoxy groups CₙH₂ₙ₊₁O with a strictly identical value of n. Said list of esters is not exhaustive. The groups R and R′ are chosen independently from a (linear, branched or cyclic) alkyl or aryl group, for example comprising between 1 and 20 carbon atoms, or even between 1 and 15 carbon atoms.

Preferably, said chemical molecule of ester type is a carboxylic ester of formula (CₙH₂ₙ₊₁O)COR with n such that n<4 or n>8, chosen from the list consisting of: methyl acetate, ethyl acetate, propyl acetate, isopropyl acetate, nonyl acetate (linear or branched), decyl acetate (linear or branched), methyl propanoate, ethyl propanoate, propyl propanoate, isopropyl propanoate, nonyl propanoate (linear or branched), decyl propanoate (linear or branched). Even more preferably, said chemical molecule of ester type is methyl acetate or methyl propanoate.

According to one or more embodiments, said chemical molecule is an ether of formula (CₙH₂ₙ₊₁O)R, with n<4 or n>8, preferably chosen from the list consisting of: dimethyl ether, diethyl ether, dipropyl ether, diisopropyl ether, dinonyl ether (linear or branched), didecyl ether (linear or branched). Preferably, said chemical molecule is dimethyl ether or diethyl ether.

A preferred ether can also be cyclopentyl methyl ether (CPME).

According to one or more other embodiments, said chemical molecule is a ketal or acetal of formula, respectively, (CₙH₂ₙ₊₁O)₂CRR′ or (CₙH₂ₙ₊₁O)₂CRH, with n<4 or n>8 and R and R′ being chosen independently from a (linear, branched or cyclic) alkyl or aryl group, said ketal or acetal chemical molecule being preferably chosen from the list consisting of: dimethylal, 2,2-dimethoxypropane, 2,2-dimethoxybutane, diethyl acetal, 2,2-diethoxypropane and 2,2-dipropoxypropane. Preferably, said chemical molecule of ketal or acetal type is dimethylal, 2,2-dimethoxypropane or 2,2-dimethoxybutane.

Preferably, step a) of solid-liquid extraction of the phthalate(s) of the PVC feedstock 1 is performed by placing said feedstock 1 in the form of particles in contact with methyl acetate or methyl propanoate, for example methyl propanoate. In this case, the DAP produced by the process is dimethyl phthalate (DMP).

Step a) of solid-liquid extraction of the phthalate(s) of the PVC feedstock 1 is preferably performed under the following operating conditions: a temperature of between room temperature and 200° C., preferably between 40° C. and 180° C., more preferably between 60° C. and 150° C., a pressure of between atmospheric pressure and 11.0 MPa, preferably between atmospheric pressure and 5.0 MPa, more preferably between atmospheric pressure and 2.0 MPa, a residence time of between 1 minute and 10 hours, preferably between 10 minutes and 4 hours, more preferably between 10 minutes and 2 hours.

Preferably, step a) is performed so that the mole ratio between the amount of the chemical molecule of ester, ether, ketal or acetal type of the solvent 9 and the amount of the phthalate(s) to be extracted from the PVC feedstock 1 is between 2 and 250, preferably between 4 and 90 and even more preferably between 4 and 30.

The reactor of step a) of the process according to the invention may advantageously be a reactor of the type stirred with a mechanical stirring system and/or with a recirculation loop and/or with fluidization, for example a perfectly stirred reactor of discontinuous or continuous type, or a reactor of rotating drum type.

As regards the implementation, the PVC feedstock in the form of particles 1 and the solvent 9 are advantageously mixed.

According to a first option, said mixing may be performed prior to the introduction of the feedstock and the solvent into the reactor of the solid-liquid extraction step a). In this case, said mixture may be formed in a mixer and may then be introduced into the reactor, said reactor being maintained at a desired pressure and temperature.

According to a second option, the PVC feedstock in the form of particles 1 and the solvent 9 may be introduced separately into the reactor of step a) of the process according to the invention. Said solid PVC feedstock and the solvent are then preferably injected into the reactor via two separate lines, one allowing the injection of the solvent 9 and the other the solid PVC feedstock in the form of particles 1. In this case, the mixture of the PVC feedstock and of the solvent forms directly in said reactor.

In accordance with the invention, said solid-liquid extraction step a) makes it possible to obtain at least one effluent 2 comprising at least a liquid phase containing at least the extracted phthalates and at least a solid phase containing the PVC plastic depleted in phthalates, preferably free of phthalates.

### Step b) of Chemical Transformation of Said Phthalates

The process according to the invention comprises a step b) of chemical transformation of the phthalate(s) extracted in step a) into at least one DAP of formula C₆H₄(COOCₙH₂ₙ₊₁)₂ by transesterification reaction, preferably in liquid phase, between said phthalate(s) of the liquid phase obtained from step a) and the chemical molecule of ester, ether, ketal or acetal type of empirical formula (CₙH₂ₙ₊₁O)ₘZ of the solvent 9, as defined above in step a), also including all the preferences of molecules as also defined in step a) for said solvent 9.

Step b) of chemical transformation of the phthalate(s) present in the liquid phase on conclusion of step a) into a DAP of formula C₆H₄(COOCₙH₂ₙ₊₁)₂ by transesterification reaction is preferably performed under the following operating conditions: a temperature of between room temperature and 200° C., preferably between 40° C. and 180° C., more preferably between 60° C. and 150° C., a pressure of between atmospheric pressure and 11.0 MPa, preferably between atmospheric pressure and 5.0 MPa, more preferably between atmospheric pressure and 2.0 MPa, a residence time of between 1 minute and 10 hours, preferably between 10 minutes and 4 hours, more preferably between 10 minutes and 2 hours. Preferably, step b) is performed so that the mole ratio between the amount of the chemical molecule of ester, ether, ketal or acetal type of the solvent 9 and the amount of the phthalate(s) to be transformed of the liquid phase containing the phthalate(s) extracted on conclusion of step a) is between 2 and 250, preferably between 4 and 90 and even more preferably between 4 and 30.

The solvent used for performing step b) is the same as that used for performing step a).

Preferably, said step b) of chemical transformation of the phthalate(s) extracted in step a) into a DAP of formula C₆H₄(COOCₙH₂ₙ₊₁)₂ by transesterification reaction is performed in the presence of a transesterification catalyst, advantageously introduced into the reaction medium.

The transesterification catalyst thus used is chosen, for example, from the catalysts of the following nonexhaustive list, which is well known to those skilled in the art, and preferably from the list consisting of:
homogeneous catalysts such as basic catalysts (sodium or potassium hydroxide, sodium or potassium methoxide, sodium or potassium carbonate, etc.), mineral Brønsted acid catalysts (hydrochloric acid, sulfuric acid, phosphoric acid, etc.), organic Brønsted acid catalysts (methanesulfonic acid, trifluoromethanesulfonic acid, trifluoroacetic acid, etc.), Lewis acid catalysts notably including boron compounds (BH₃, BF₃) and aluminum compounds (AlF₃, AlCl₃), and organometallic compounds;
heterogeneous catalysts such as alkaline-earth metal oxides (CaO, BaO, etc.), alkali metal and/or alkaline-earth metal carbonates or hydrogen carbonates (CaCO₃, etc.), alkali metals supported on aluminas or zeolites, zinc oxides and mixtures thereof with other oxides (for example zinc oxide and alumina), ion-exchange resins (cations or anions), for instance sulfonic resins, etc.

For example, the catalyst used according to the invention is a homogeneous catalyst, notably a homogeneous catalyst of basic catalyst type such as sodium methoxide.

When the chemical molecule is an ester as defined above at step a), in particular an ester of formula (CₙH₂ₙ₊₁O)COR with n such that n<4 or n>8, preferably chosen from the list consisting of: methyl acetate, ethyl acetate, propyl acetate, isopropyl acetate, nonyl acetate (linear or branched), decyl acetate (linear or branched), methyl propanoate, ethyl propanoate, propyl propanoate, isopropyl propanoate, nonyl propanoate (linear or branched), decyl propanoate (linear or branched), for example methyl acetate or ethyl acetate, a preferred catalyst is a homogeneous catalyst, especially a homogeneous catalyst of basic type like sodium methoxide.

When the chemical molecule is an ether as defined above at step a), in particular an ether of formula (CₙH₂ₙ₊₁O)R, with n<4 or n>8, preferably chosen from the list consisting of: dimethyl ether, diethyl ether, dipropyl ether, diisopropyl ether, dinonyl ether (linear or branched), didecyl ether (linear or branched), and cyclopentyl methyl ether, for example dimethyl ether or diethyl ether, a preferred catalyst is a homogeneous catalyst, especially an acid catalyst, typically an organic Brønsted acid catalyst like methanesulfonic acid, trifluoromethanesulfonic acid, or trifluoroacetic acid.

When the chemical molecule is a ketal or acetal as defined above at step a), in particular a ketal or acetal of formula, respectively, (CₙH₂ₙ₊₁O)₂CRR′ or (CₙH₂ₙ₊₁O)₂CRH, with n<4 or n>8 and R and R′ being chosen independently from a (linear, branched or cyclic) alkyl or aryl group, said ketal or acetal chemical molecule being preferably chosen from the list consisting of: dimethylal, 2,2-dimethoxypropane, 2,2-dimethoxybutane, diethyl acetal, 2,2-diethoxypropane and 2,2-dipropoxypropane, for example dimethylal, a preferred catalyst is a homogeneous catalyst, especially an acid catalyst, typically an organic Brønsted acid catalyst like methanesulfonic acid, trifluoromethanesulfonic acid, or trifluoroacetic acid.

Preferably, the amount of catalyst introduced is such that the mass ratio between the catalyst and the phthalate(s) to be transformed is between 0.5% and 15% by mass, preferably between 1% and 10% by mass and even more preferably between 1% and 8% by mass.

Whether it is homogeneous or heterogeneous, the catalyst may be recycled and/or removed in the process according to methods that are well known to those skilled in the art, and is preferably recycled. It may be isolated, to be removed or preferably recycled for the transesterification reaction, in the downstream steps of the process, for example in step c), in step d) and/or in step e), or in any other dedicated step.

The reactor of step b) of the process according to the invention may advantageously be a reactor of the type stirred with a mechanical stirring system and/or with a recirculation loop and/or with fluidization, for example a perfectly stirred reactor of discontinuous or continuous type, or a reactor of rotating drum type.

In accordance with the invention, said step b) of transformation of the phthalates makes it possible to obtain at least one effluent comprising at least one liquid phase containing at least the DAP of formula C₆H₄(COOCₙH₂ₙ₊₁)₂ obtained after transesterification reaction, i.e. the liquid phase formed on conclusion of step a) and enriched in DAP in step b).

Steps a) and b) of the process according to the invention may be performed in the same individual operation or may be the subject of two distinct and consecutive individual operations, the individual operation of step a) then always being performed prior to the individual operation of step b).

In the embodiments presented in FIGS. 1 to 5, steps a) and b), although featured in the form of separate “boxes”, may be performed either in the same individual operation, or may be the subject of two distinct and consecutive individual operations. In the first case, the effluent 2 is present in the same reactor used, for example, for performing the two steps a) and b).

In the embodiment presented in FIG. 6, which is one of the preferred embodiments according to the invention, steps a) and b) are the subject of the same individual operation, which this time is featured by the use of a single “box” (a+b).

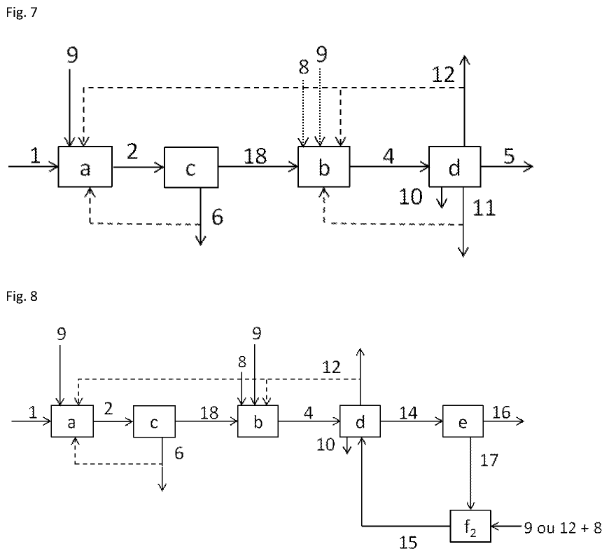

In the embodiments represented in FIGS. 7 and 8, that of FIG. 8 being one of the preferred embodiments according to the invention, steps a) and b) form the subject of two distinct and consecutive individual operations, corresponding to a scheme in which step c) is performed between steps a) and b), as described below.

### Step c) of Solid-Liquid Separation

The process according to the invention comprises a step c) of solid-liquid separation between, on the one hand, the liquid phase containing the phthalate(s) extracted in step a) and/or the DAP of formula C₆H₄(COOCₙH₂ₙ₊₁)₂ obtained after transesterification reaction in step b), and, on the other hand, the solid phase containing the PVC plastic depleted in phthalates, preferably free of phthalates.

The physical separation of the liquid phase and of the solid phase may advantageously be performed according to the techniques known to those skilled in the art, such as, in a nonexhaustive manner, filtration, centrifugation, electrostatic precipitation or decantation, said techniques being used alone or in combination, in any order.

This step c) of solid-liquid separation thus makes it possible to produce at least one solid stream (6) including the PVC plastic depleted in the phthalate(s) extracted in step a), so as to recover said reusable target PVC plastic.

The production of the reusable target PVC as defined according to the invention may necessitate returning all or a portion of the solid stream (6) obtained in step c) into step a), in as many cycles as necessary so as to produce said target PVC plastic.

This possibility of recycling the solid stream is shown in FIGS. 2 to 8.

According to a first variant of the process according to the invention, said solid-liquid separation step c) takes place after performing steps a) and b). This first variant is illustrated in FIGS. 1 to 6. In this case, the liquid effluent 3 obtained from step b) is sent into the solid-liquid separation step c) which leads to separation between the liquid phase, containing at least the DAP obtained after transesterification reaction in step b), and the solid phase containing the PVC plastic depleted in phthalate(s). Advantageously for this first variant of the process according to the invention, steps a) and b) are performed together in the same individual operation, this specific implementation leading to a reduction in the number of individual operations required for performing the process according to the invention and thus to a limitation of the number of items of equipment, of the amount of solvent used, of the energy engaged, etc., and thus a reduction of the costs. A preferred example of implementation according to this variant is illustrated in FIG. 6.

According to a second variant of the process according to the invention, the solid-liquid separation step c) takes place after performing step a) and before performing b). This second variant is illustrated in particular in FIGS. 7 and 8. In this case, the liquid effluent 2 obtained from step a) is sent into the solid-liquid separation step c) which leads to separation of the liquid phase containing the extracted phthalates from the solid phase containing the PVC depleted in phthalate(s). Consequently, for this second variant, steps a) and b) form the subject of two distinct individual operations. Step c) thus produces the solid stream 6 including the PVC plastic depleted in phthalate(s), and a first liquid stream 18 which contains the phthalate(s) extracted in step a) and which is then sent into step b) for transformation of said phthalate(s) by transesterification. This second variant is particularly suitable in the case where the PVC feedstock to be treated would lead to the formation, during step a), of a solid phase that is unfavorable for performing the transesterification chemical reaction (in terms of chemical or rheological properties, etc.).

For example, according to embodiments in accordance with this second variant of the process as shown in FIGS. 7 and 8, in which step c) is performed between steps a) and b), steps a) and c) according to the invention may be consecutively performed in the same discontinuous reactor having a device for filtering the liquid effluent 2 allowing several cycles of extraction of the phthalates from the solid phase and a device for withdrawing at least the solid phase 6 allowing the final recovery of the target PVC plastic.

For another example, step c) may take place by centrifugation of the liquid effluent 2 or 3 comprising the liquid phase containing at least the extracted phthalates and/or the DAP and of the solid phase obtained from step a), leading to the separation of said solid 6, and advantageously to the return of all or a portion of said solid into step a), preferably placed in suspension beforehand, for example by means of supplying solvent 9 (not shown in the figures), until the reusable target PVC plastic is produced.

### Step d) of Liquid-Liquid Separation

The process according to the invention comprises a liquid-liquid separation step d) for extracting the DAP of formula C₆H₄(COOCₙH₂ₙ₊₁)₂ from the liquid phase obtained on conclusion of the implementation of at least the steps a), b) and c).

A liquid stream (4, 13) containing said liquid phase advantageously feeds this liquid-liquid separation step d), which thus makes it possible to produce at least a first liquid effluent including the DAP (stream 5 or 14 according to the figures) and a second liquid effluent comprising at least said solvent (stream 7 or 12 according to the figures).

The liquid-liquid separation step d) may be performed according to methods that are well known to those skilled in the art, such as, in a nonexhaustive manner, distillation, decantation, evaporation, liquid-liquid extraction, etc., performed alone or in combination. The operating conditions of this step (temperature, pressure, etc.) are determined as a function of the chosen separation method.

According to one or more embodiments, the first effluent 5 consists essentially of said DAP. In this case (these cases), the second liquid effluent 7, represented, for example, in FIG. 1 (or as an option in FIG. 3), consists of the residual liquid phase after extraction of the DAP, which contains at least the solvent, the byproducts of ester, ether, ketal or acetal type (BP), the intermediate alkyl phthalates (IAP) and the phthalate(s) extracted on conclusion of step a) of the process according to the invention which are possibly not converted. The second liquid effluent 7 may be returned, totally or partly, preferably totally, into step b) of the process according to the invention.

It is also possible in this case (these cases), notably depending on the chosen liquid-liquid separation methods, for example distillation with side withdrawal or liquid-liquid extraction, to separate from the liquid phase not only the solvent, but also the BPs and very advantageously the IAPs optionally with the unconverted phthalates extracted in step a). Such a separation is illustrated, for example, in FIG. 2 or in FIG. 7 (and as an alternative to the production of a stream 7 in FIG. 3), in which it may be seen that step d) produces, in addition to the first effluent 5 consisting essentially of said DAP and the second effluent 12 consisting essentially of said solvent, a third effluent 10 including BPs obtained during the transesterification in step b), and a fourth effluent 11 including phthalate(s) that are partially converted (IAP) and/or unconverted in step b) and possibly other soluble impurities. The fourth effluent 11 may then be advantageously returned into step b) of the process according to the invention, notably according to the first and second variants of the process according to the invention, so as to continue the chemical reactions leading to the DAP and thus to improve the yield of this product.

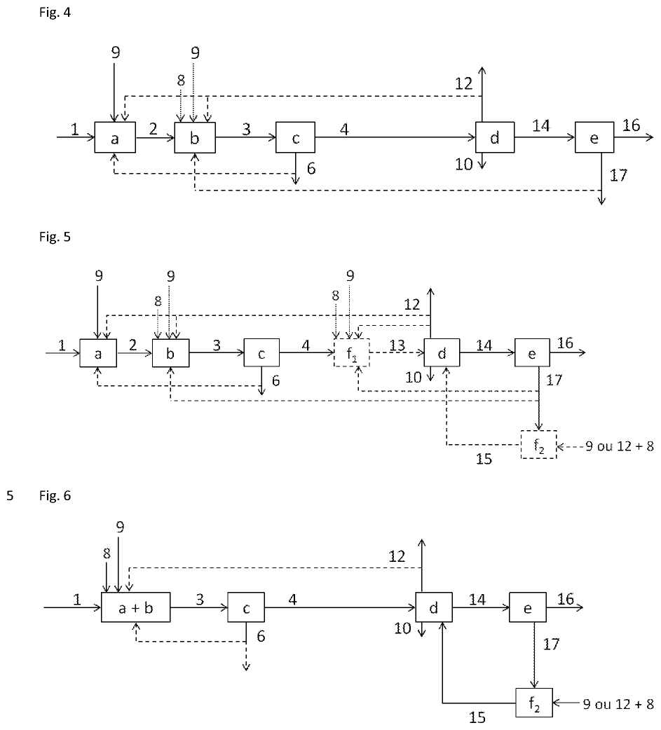

According to one or more alternative embodiments, as shown in FIGS. 4 to 6 and in FIG. 8, the first liquid effluent 14 including the DAP also comprises other compounds such as phthalate(s) that are partially converted (IAP) and/or unconverted in step b) and/or soluble impurities (for example IAPs). As described later, according to this or these embodiments, a step of purification of the DAP of the first effluent is necessary. According to this or these embodiments, the liquid-liquid separation step d) thus advantageously produces said first liquid effluent 14 of impure DAP, a second liquid effluent 12 preferably consisting essentially of said solvent, and preferably a third effluent 10 including BPs obtained during the transesterification in step b). Isolation of the BPs and of the solvent is notably made possible as a function of the liquid-liquid separation methods chosen, for instance distillation with side withdrawal or liquid-liquid extraction. In the case where the second liquid effluent 12 consists essentially of said solvent thus recovered, the second liquid effluent 12 may then be advantageously returned, partly or totally, preferably totally, into step a) and/or step b) of the process according to the invention, and notably according to the first and second process variants according to the invention.

### Step e) of Purification of the DAP (Optional)

The process according to the invention may comprise an optional step e) of purification of the first effluent 14 comprising the DAP obtained from the liquid-liquid separation step d), to improve its quality and thus, ultimately, its upgrading. The embodiments shown in FIGS. 4, 5, 6 and 8 illustrate the implementation of such a purification step e).

In the case of performing said step e), the solvent was advantageously isolated during the implementation of step d). Moreover, the IAPs and optionally the phthalate(s) extracted on conclusion of step a) of the process according to the invention and not converted on conclusion of step b) may have been isolated during step d) of the process according to the invention, or alternatively may be isolated during the implementation of said purification step e).

Thus, it is possible to send the first effluent 14, comprising the DAP, phthalate(s) which are partially converted and/or unconverted in step b) and possibly soluble impurities, into this purification step e) to form a liquid product 16 consisting essentially of said DAP, and a liquid residue 17 comprising the phthalate(s) which are partially converted and/or unconverted in step b) and possibly the soluble impurities.

The liquid residue 17 thus recovered may then be advantageously returned into step b) of the process according to the invention, notably according to the first and second variants of the process according to the invention, so as to continue the chemical reactions leading to the DAP, as illustrated in FIG. 4 or in FIG. 5.

The purification step e) may advantageously be performed via methods that are well known to those skilled in the art, such as precipitation, crystallization or adsorption, optionally followed by filtration or centrifugation. The purification step e) may comprise the implementation of several of these methods in parallel or in series. For example, and without being exhaustive, the purification step e) may comprise a precipitation and filtration step, followed by an adsorption step, or alternatively may comprise an adsorption and filtration step, optionally followed by a precipitation step, or may alternatively comprise a crystallization and filtration step. The operating conditions of this step e) (temperature, pressure, etc.) are determined as a function of the chosen purification method.

Additional Step(s) f₁) and/or f₂) of Chemical Transformation by Transesterification (Optional)

In order to promote the production of the DAP according to the invention, it is possible to perform, independently of step b) of chemical transformation of the phthalate(s) extracted in step a), an additional chemical transformation step allowing the transformation of the IAPs and/or of the extracted phthalate(s) which are possibly not converted on conclusion of step b).

The process may thus also comprise an additional step f₁), as shown in FIG. 3 or FIG. 5, of chemical transformation by transesterification of the phthalate(s) which are unconverted in step b) and/or of at least one IAP produced in step b), into DAP of formula C₆H₄(COOCₙH₂ₙ₊₁)₂ using the solvent. In these embodiments, step f₁) is performed between steps c) and d), and advantageously after step b), by sending the liquid phase 4, advantageously obtained on conclusion of all the steps a), b) and c), into a first additional transesterification reactor, to produce a second liquid stream 13 enriched in DAP, said second liquid stream 13 being sent into step d). According to this embodiment, step c) is preferably performed on conclusion of step b).

The process may also comprise an additional step f₂) of chemical transformation by transesterification of the unconverted phthalate(s) in step b) and/or of at least one IAP produced in step b) or optionally in the optional step f₁), into DAP of formula C₆H₄(COOCₙH₂ₙ₊₁)₂ using the solvent, step f₂) being performed after step e) by sending the liquid residue 17 into a second additional transesterification reactor to produce a third liquid stream 15 enriched in said DAP, said third liquid stream 15 being returned into step d).

The implementation of the additional step f₁) and/or of the additional step f₂) of chemical transformation by transesterification may be performed according to the first variant (solid-liquid separation step c) performed after steps a) and b)) or the second variant (solid-liquid separation step c) between steps a) and b)) of the process according to the invention.

Preferably, the process according to the invention comprises only one additional step of chemical transformation by transesterification, and preferably step f₂).

The implementation of step f₁) and/or of step f₂) is as described for step b) of the process according to the invention. In particular, the ranges associated with the operating conditions of steps b) and f₁) and/or f₂) are similar, and said ranges are chosen by a person skilled in the art so as to promote the production of the DAP as a function of the chemical nature of the stream to be treated at the inlet of said step f₁) and/or step f₂).

This is likewise the case for the preferred use of a transesterification catalyst 8, as described in step b). The transesterification catalyst in step(s) f₁) and/or f₂) may be identical to or different from the one used in step b).

Said stream sent into step f₁) and/or step f₂) (stream 4 or liquid residue 17) is a liquid phase comprising one or more phthalates extracted in step a) and which are possibly partially converted (IAP) and/or unconverted in step b), and possibly soluble impurities, which are then isolated either during the implementation of the liquid-liquid separation step d) of the process according to the invention, or during the implementation of the purification step e) of the process according to the invention if said step is advantageously performed.

Depending on the sequence of steps considered involving step f₁) and/or step f₂), it may be necessary to use an additional supply of solvent, this additional supply of solvent possibly resulting from a supply of “fresh” solvent 9 or else from recycling of the stream 12 of said solvent optionally isolated on conclusion of step d) of the process according to the invention. This additional supply in the first additional transesterification reactor performed in step f₁) and/or in the second additional transesterification reactor performed in step f₂), by supplying fresh solvent 9, and/or by recycling the second effluent 12 consisting of said solvent, is illustrated in FIGS. 3, 5, 6 and 8.

When the purification step e) is performed, at least a portion of said liquid residue 17 produced in step e) may be recycled into step f₁), as illustrated in FIG. 5, so as to continue the chemical reactions leading to the DAP.

FIGS. 6 and 8 represent preferred embodiments according, respectively, to the first variant (solid-liquid separation step c) after performing steps a) and b)), and to the second variant (solid-liquid separation step c) between steps a) and b)) of the process according to the invention.

As may be seen in FIG. 6, according to a preferred embodiment of the invention in accordance with the first variant, the process includes an implementation in the same individual operation of steps a) and b), a solid-liquid separation step c) after steps a) and b), a liquid-liquid separation step d), a step e) of purification of a first effluent 14 obtained in step d) comprising the DAP, and advantageously an additional step of transesterification f₂) of the residue 17 obtained from step e).

According to this embodiment, as illustrated schematically in FIG. 6, the PVC feedstock in the form of particles 1, optionally preconditioned, is introduced into a reactor combining the implementation of steps a) and b) of, respectively, solid-liquid extraction and chemical transformation by transesterification preferably in the presence of a catalyst 8. The reactor is also fed with a stream of fresh solvent 9 external to the process, comprising at least, and preferably consisting of, a chemical molecule of ester, ether, ketal or acetal type of empirical formula (CₙH₂ₙ₊₁O)ₘZ as defined previously, the chemical molecule being, for example, methyl acetate or methyl propanoate, preferably methyl propanoate, and optionally with at least a fraction of a stream 12 of solvent isolated in the liquid-liquid separation step d). The reaction effluent 3 containing the liquid phase including at least the DAP, preferably DMP, and the solid phase including the PVC plastic depleted in phthalates, preferably free of phthalates, is sent into the solid-liquid separation step c), for example performing a centrifugation, to produce a solid stream 6 including said PVC plastic depleted in the extracted phthalate(s) so as to recover said reusable target PVC plastic, and a liquid stream 4 containing at least the DAP, preferably DMP, and at least the solvent. The solid stream 6 may be partly recycled into step a). The liquid stream 4 obtained from step c), containing the DAP, the solvent, possibly the unconverted or partially converted (IAP) phthalate(s) and possibly byproducts (BPs), is sent into the liquid-liquid separation step d), which makes it possible to isolate, on the one hand, the solvent as a stream 12, but also, preferably, the BPs as a stream 10, and finally a liquid effluent 14 including the DAP, preferably DMP, and possibly partially converted and/or unconverted phthalate(s) and possibly soluble impurities. The liquid effluent 14 is sent into a purification step e), so as to obtain the purified DAP, preferably DMP. Since the residue 17 obtained from this purification step e) may still contain unconverted or partially converted (IAP) phthalate(s), an additional chemical transformation step f₂) of transesterification is preferably performed. The residue 17 is thus advantageously sent into a second transesterification reactor containing a suitable transesterification catalyst, to perform the transesterification of the unconverted or partially converted (IAP) phthalate(s) using a solvent 9. The solvent may be a supply of fresh solvent or may originate from the stream 12 at least partly recycled into this step f₂). This step f₂) produces a liquid stream 15 enriched in said DAP, preferably in DMP, which is returned into the liquid-liquid separation step d).

As shown in FIG. 8, according to another preferred embodiment of the invention in accordance with the second variant, the process includes an implementation of steps a) and b) in two distinct individual operations, with a step c) performed between steps a) and b), followed by a step d), and also includes a step e) of purification of a first effluent 14 obtained in step d) comprising the DAP, and an additional step of transesterification f₂) of the residue 17 obtained from step e).

According to this embodiment, as illustrated schematically in FIG. 8, the PVC feedstock in the form of particles 1, optionally preconditioned, is introduced into a reactor to perform step a) of solid-liquid extraction of the phthalate(s) from said PVC feedstock. The reactor is fed with a stream of fresh solvent 9 external to the process, comprising a chemical molecule of ester, ether, ketal or acetal type, for example methyl acetate or methyl propanoate, preferably methyl propanoate, and optionally with a stream 12 of solvent isolated in the subsequent liquid-liquid separation step d). The effluent 2 produced in step a) comprises at least a liquid phase containing at least the phthalate(s) extracted from said feedstock 1 and at least a solid phase containing the PVC plastic depleted in phthalates, preferably free of the extracted phthalates. The effluent 2 is sent into a solid-liquid separation step c), for example performing a centrifugation, to produce a solid stream 6 including said PVC plastic depleted in phthalate(s), so as to recover said reusable target PVC plastic, and a liquid stream 18 containing at least the phthalate(s) extracted in step a), and at least the solvent. The liquid stream 18 is then sent into a reactor to perform step b) of chemical transformation of the extracted phthalate(s) by transesterification, preferably in the presence of a catalyst 8. The transesterification reactor may also be fed with a stream of fresh solvent 9 external to the process, comprising the same chemical molecule of ester, ether, ketal or acetal type, for example methyl acetate or methyl propanoate, preferably methyl propanoate, and optionally with at least a fraction of a stream 12 of solvent isolated in the liquid-liquid separation step d). The reaction effluent 4 containing the liquid phase including at least the DAP, preferably DMP, the solvent, possibly the unconverted or partially converted (IAP) phthalate(s) and possibly byproducts (BPs), is sent into the liquid-liquid separation step d), which makes it possible to isolate, on the one hand, the solvent as a stream 12, but also the BPs as a stream 10, and finally a liquid effluent 14 including the DAP, preferably DMP, and possibly partially converted (IAP) and/or unconverted phthalate(s) and possibly soluble impurities. The liquid effluent 14 is preferably sent into a purification step e), so as to obtain the purified DAP 16, preferably DMP. Since the residue 17 obtained from this purification step e) may still contain unconverted or partially converted (IAP) phthalate(s), an additional transesterification chemical transformation step f₂) is preferably performed. The residue 17 is advantageously sent into a second transesterification reactor preferably containing a suitable transesterification catalyst, to perform the transesterification of the unconverted or partially converted (IAP) phthalate(s) using a solvent 9 comprising the chemical molecule of ester, ether, ketal or acetal type of empirical formula (CₙH₂ₙ₊₁O)ₘZ as defined previously. The solvent may be a supply of fresh solvent or may originate from the stream 12 at least partly recycled into this step f₂). This step f₂) produces a liquid stream 15 enriched in said DAP, preferably in DMP, which is returned into the liquid-liquid separation step d).

The present invention also relates to a process for recycling a PVC-based object containing at least one phthalate, said recycling process including:
the conditioning of the PVC-based object comprising at least milling or shredding of the PVC-based object to form a PVC feedstock in the form of particles;
the recovery of a DAP and of a reusable target PVC plastic from said PVC feedstock in the form of particles according to the process, described above in detail, for the recovery of a DAP and of a reusable target PVC plastic.

The step of conditioning of the PVC-based object may include the various steps detailed above for the preconditioning of the PVC feedstock before it is introduced into step a).

The present invention also relates to a process for manufacturing a flexible PVC-based object including a recycled PVC plastic and/or a DAP which are obtained via the process for recovering a DAP and a reusable target PVC plastic described above in detail.

Such a manufacturing process typically comprises a step of recovering a DAP and a reusable target PVC plastic from a PVC feedstock, as detailed above, followed by a step of mixing said reusable target PVC plastic with additives or a step of mixing said recovered DAP with a PVC resin, and then a step of forming said mixture.

### EXAMPLES

### Example 1

This example 1 illustrates the invention without limiting the scope thereof, and notably illustrates the extraction of a phthalate contained in a PVC plastic and the conversion of the phthalate into dimethyl phthalate in the presence of a catalyst, by transesterification using the carboxylic ester chemical molecule methyl propanoate.

18.2 g of a PVC plastic feedstock (obtained from PVC-based objects of “medical tubing” type), in the form of extrudates with a mean size of 2 mm, containing 4.4 g of didecyl phthalate (DIDP), are introduced into a reactor stirred with a mechanical stirring system, of paddle type. 17.35 g of methyl propanoate are then added, the methyl propanoate/DIDP mole ratio being 20. 0.17 g of catalyst (NaOMe) are then added to the preceding mixture so that the NaOMe/DIDP mass percentage is 4%.

The reactor is hermetically closed, purged with nitrogen and then heated to 100° C. with an autogenous pressure of the order of 1.2 MPa and maintained under these conditions for 4 hours with stirring of 1000 rpm. The reactor is then cooled.

After 4 hours, a solid and a liquid are obtained, and are analyzed.

The analyses by gas chromatography with flame ionization detection (GC-FID) of the liquid phase show that it contains 0.13 g of dimethyl phthalate (DMP) obtained from the conversion of the DIDP and 0.07 g of decyl methyl phthalate due to a partial conversion of the DIDP. Identification was made possible by comparison of the retention times of pure analytical standards and quantification was performed by determination of the response coefficients derived from the analysis of these same standards.

The solid obtained was prefractionated by preparative size exclusion chromatography SEC equipped with double optical detection (UV/visible) and refractometry (RI). The fractions obtained from the collection were analyzed by high-performance liquid chromatography (HPLC) equipped with optical detection of quantitative UV-visible type The results indicate the presence of DIDP in the target PVC plastic in a content of less than 1000 ppm, which complies with the European regulations in force.

These results show that a phthalate-free PVC in accordance with the invention is obtained, and that the DIDP was 10% converted. This degree of conversion takes into account the total conversion of the DIDP into DMP and its partial conversion. The unconverted DIDP is in the liquid phase. In this example, the extraction of the DIDP and its conversion are performed in the same step.

### Example 2

This example 2 illustrates the invention without limiting the scope thereof, and notably illustrates the extraction of a phthalate contained in a PVC plastic and the conversion of the phthalate into dimethyl phthalate in the presence of a catalyst, by transesterification using the carboxylic ester chemical molecule methyl acetate.

13.23 g of a PVC plastic feedstock (obtained from PVC-based objects of “medical tubing” type), in the form of extrudates with a mean size of 2 mm, containing 3.2 g of didecyl phthalate (DIDP), are introduced into a reactor stirred with a mechanical stirring system, of paddle type. 10.61 g of methyl acetate are then added, the methyl acetate/DIDP mole ratio being 20. 0.12 g of catalyst (NaOMe) are then added to the preceding mixture so that the NaOMe/DIDP mass percentage is 4%.

The reactor is hermetically closed, purged with nitrogen and then heated to 100° C. with an autogenous pressure of the order of 1.2 MPa and maintained under these conditions for 4 hours with stirring of 1000 rpm. The reactor is then cooled.

After 4 hours, a solid and a liquid are obtained, and are analyzed.

The analyses by gas chromatography with flame ionization detection (GC-FID) of the liquid phase show that it contains 0.07 g of dimethyl phthalate (DMP) obtained from the conversion of the DIDP and 0.17 g of decyl methyl phthalate due to a partial conversion of the DIDP. Identification was made possible by comparison of the retention times of pure analytical standards and quantification was performed by determination of the response coefficients derived from the analysis of these same standards.

The solid obtained was prefractionated by preparative size exclusion chromatography SEC equipped with double optical detection (UV/visible) and refractometry (RI). The fractions obtained from the collection were analyzed by high-performance liquid chromatography (HPLC) equipped with optical detection of quantitative UV-visible type The results indicate the presence of DIDP in the target PVC plastic in a content of less than 1000 ppm, which complies with the European regulations in force.

These results show that a phthalate-free PVC in accordance with the invention is obtained, and that the DIDP was 9% converted. This degree of conversion takes into account the total conversion of the DIDP into DMP and its partial conversion. The unconverted DIDP is in the liquid phase. In this example, the extraction of the DIDP and its conversion are performed in the same step.

1. A process for recovering a dialkyl phthalate and a reusable target PVC plastic from a PVC feedstock containing at least one phthalate, said process comprising:
a) solid-liquid extraction of said PVC feedstock in the form of particles (1) by placing said particles of the PVC feedstock in contact with a solvent (9) including at least one chemical molecule of ester, ether, ketal or acetal type, of empirical formula (CₙH₂ₙ₊₁O)ₘZ, n and m being positive integers with n<4 or n>8, m greater than or equal to 1 and less than or equal to 3, and Z being a group chosen from the list consisting of the following elements: R, COOR, CO, CR, CNRR′, PO, P, SO, SO2, COR, and HCO, with R and R′ chosen independently from a linear, branched or cyclic alkyl group, or an aryl group, to produce a liquid phase enriched in said phthalate and a solid phase including PVC plastic depleted in said phthalate;
b) chemical transformation of said phthalate of said liquid phase into dialkyl phthalate of formula C₆H₄(COOCₙH₂ₙ₊₁)₂ by transesterification using said chemical molecule of ester, ether, ketal or acetal type of empirical formula (CₙH₂ₙ₊₁O)ₘZ to enrich said liquid phase in said dialkyl phthalate;
c) a solid-liquid separation between said solid phase and said liquid phase to produce at least one solid stream including the PVC plastic depleted in said phthalate (6) so as to recover said target PVC plastic; and
d) a liquid-liquid separation of said liquid phase, to produce at least a first liquid effluent including said dialkyl phthalate (5, 14) and a second liquid effluent comprising at least said solvent (7, 12).

2. The process as claimed in claim 1, in which steps a) and b) are performed within the same individual operation.

3. The process as claimed in claim 1, in which steps a) and b) form the subject of two distinct operations, step a) producing a stream including said liquid phase and said solid phase (2).

4. The process as claimed in claim 3, in which step c) is performed between steps a) and b), said stream including said liquid phase and said solid phase (2) obtained from step a) being sent into the solid-liquid separation step c) to produce said stream including the PVC plastic depleted in said phthalate (6) and a first liquid stream (18) including said liquid phase sent into step b).

5. The process as claimed in claim 1, further comprising an additional step f₁) of chemical transformation by transesterification of said phthalate which is unconverted and/or partially converted in step b), into dialkyl phthalate of formula C₆H₄(COOCₙH₂ₙ₊₁)₂ by means of said solvent (9), said step f₁) being performed between steps c) and d) by sending said liquid phase obtained on conclusion of all the steps a), b) and c) into a first additional transesterification reactor to produce a second liquid stream (13) enriched in said dialkyl phthalate of formula C₆H₄(COOCₙH₂ₙ₊₁)₂, said second liquid stream (13) being sent into step d).

6. The process as claimed in claim 5, in which said solvent (9) is supplied and/or at least a portion of said second liquid effluent comprising at least said solvent (7, 12) obtained from step d) is recycled into the first additional transesterification reactor.

7. The process as claimed in claim 1, wherein, in step d), said first effluent (5) consists essentially of said dialkyl phthalate.

8. The process as claimed in claim 1, wherein the liquid-liquid separation step d) also produces a third effluent (10) including byproducts of ester, ether, ketal or acetal type obtained during step b) and optionally a fourth effluent (11) including said phthalate that is partially converted and/or unconverted in step b) and optionally other soluble impurities, said first liquid effluent (5) consisting essentially of said dialkyl phthalate and said second liquid effluent (12) consisting essentially of said solvent.

9. The process as claimed in claim 1, wherein:
the liquid-liquid separation step d) also produces a third effluent (10) including byproducts of ester, ether, ketal, or acetal type obtained during step b), said first liquid effluent (14) comprising said dialkyl phthalate, phthalate that is partially converted and/or unconverted in step b) and optionally soluble impurities, said second liquid effluent (12) consisting essentially of said solvent, and
said process further comprises:
e) purification of said first liquid effluent (14) to produce a liquid product (16) consisting essentially of said dialkyl phthalate, and a liquid residue (17) comprising said phthalate that is partially converted and/or unconverted in step b) and optionally said soluble impurities.

10. The process as claimed in claim 9, also comprising an additional step f₂) of chemical transformation by transesterification of said phthalate which is unconverted and/or partially converted in step b), into dialkyl phthalate of formula C₆H₄(COOCₙH₂ₙ₊₁)₂ by means of said solvent (9), said step f₂) being performed after step e) by sending said liquid residue (17) into a second additional transesterification reactor to produce a third liquid stream (15) enriched in said dialkyl phthalate of formula C₆H₄(COOCₙH₂ₙ₊₁)₂, said third liquid stream (15) being returned into step d).

11. The process as claimed in claim 10, in which said solvent (9) is supplied and/or at least a portion of said second liquid effluent comprising at least said solvent (12) obtained from step d) is recycled into the second additional transesterification reactor.

12. The process as claimed in claim 9, further comprising the recycling of at least a portion of said liquid residue (17) into step b) and/or an additional step f₁) of chemical transformation by transesterification of said phthalate which is unconverted and/or partially converted in step b), into dialkyl phthalate of formula C₆H₄(COOCₙH₂ₙ₊₁)₂ by means of said solvent (9), said step f₁) being performed between steps c) and d) by sending said liquid phase obtained on conclusion of all the steps a), b) and c) into a first additional transesterification reactor to produce a second liquid stream (13) enriched in said dialkyl phthalate of formula C₆H₄(COOCₙH₂ₙ₊₁)₂, said second liquid stream (13) being sent into step d).

13. The process as claimed in claim 1, wherein said second liquid effluent (7, 12) comprising at least said solvent (9) obtained from step d) is at least partly recycled into step a) and/or step b).

14. The process as claimed in claim 1, wherein said solid stream including the PVC plastic depleted in phthalates (6) is at least partly recycled into step a).

15. The process as claimed in claim 1, wherein said chemical molecule of said solvent (9) is an ester bearing one or more alkoxy groups of formula (CₙH₂ₙ₊₁O)ₘ, with n<4 or n>8 and m greater than or equal to 1 and less than or equal to 3, said ester being preferably chosen from the list consisting of the carboxylic esters of formula (CₙH₂ₙ₊₁O)COR, the carbonate esters of formula (CₙH₂ₙ₊₁O)₂CO, the orthoesters of formula (CₙH₂ₙ₊₁O)₃CR, the imino esters of formula (CₙH₂ₙ₊₁O)CNRR′, the phosphite esters of formula (CₙH₂ₙ₊₁O)₃P, the phosphate esters of formula (CₙH₂ₙ₊₁O)₃PO, the sulfite esters of formula (CₙH₂ₙ₊₁O)₂SO, the sulfate esters of formula (CₙH₂ₙ₊₁O)₂SO₂, and mixtures thereof, with the proviso that the esters involved in said mixtures contain alkoxy groups CₙH₂ₙ₊₁O with a value of n that is strictly identical, and more preferentially said chemical molecule of said solvent (9) is a carboxylic ester of formula (CₙH₂ₙ₊₁O)COR with n<4 or n>8, preferably chosen from the list consisting of methyl acetate, ethyl acetate, propyl acetate, isopropyl acetate, nonyl acetate, which may be linear or branched, decyl acetate, which may be linear or branched, methyl propanoate, ethyl propanoate, propyl propanoate, isopropyl propanoate, nonyl propanoate, which may be linear or branched, and decyl propanoate, which may be linear or branched, and preferably from methyl acetate and methyl propanoate.

16. The process as claimed in claim 1, wherein said chemical molecule of said solvent (9) is an ether of formula (CₙH₂ₙ₊₁O)R, with n<4 or n>8, preferably chosen from the list consisting of dimethyl ether, diethyl ether, dipropyl ether, diisopropyl ether, dinonyl ether, which may be linear or branched, didecyl ether, which may be linear or branched, and cyclopentyl methyl ether, and more preferentially from dimethyl ether and diethyl ether.

17. The process as claimed in claim 1, wherein said chemical molecule of said solvent (9) is a ketal or acetal of formula, respectively, (CₙH₂ₙ₊₁O)₂CRR′ or (CₙH₂ₙ₊₁O)₂CRH, with n<4 or n>8, preferably chosen from the list consisting of dimethylal, 2,2-dimethoxypropane, 2,2-dimethoxybutane, diethyl acetal, 2,2-diethoxypropane and 2,2-dipropoxypropane, and more preferentially from dimethylal, 2,2-dimethoxypropane and 2,2-dimethoxybutane.

18. The process as claimed in claim 1, wherein said chemical molecule of said solvent (9) is methyl propanoate and said dialkyl phthalate is dimethyl phthalate.

19. The process as claimed in claim 1, wherein the chemical transformation performed by transesterification in step b), and optionally in step f₁) and/or f₂), is performed in the presence of a catalyst preferably chosen from the list consisting of mineral or organic basic or acidic Brønsted homogeneous catalysts, or Lewis acids, and heterogeneous catalysts formed by alkaline-earth metal oxides, or alkali metal and/or alkaline-earth metal carbonates or hydrogen carbonates, or alkali metals supported on aluminas or zeolites, or zinc oxides and mixtures thereof with other oxides, or ion-exchange resins.

20. The process as claimed in claim 1, wherein said at least one phthalate of said PVC feedstock is a phthalate of empirical formula C₆H₄(COOR₁)(COOR₂) in which the ester groups are in the ortho position of the benzene nucleus, R₁ or R₂ being chosen independently from one of the elements of the group consisting of a linear or branched or cyclic alkyl chain, a linear or branched alkoxyalkyl chain, or an aryl or alkylaryl chain, R₁ and/or R₂ preferably comprising between 1 and 20 carbon atoms, or even between 1 and 15 carbon atoms.

21. The process as claimed in claim 1, wherein said target PVC plastic is substantially free of said phthalate, and preferably comprises less than 0.1% by mass in total of phthalates chosen from the list consisting of dibutyl phthalate, dioctyl phthalate or diethylhexyl phthalate, benzyl butyl phthalate, dibutyl phthalate, diisobutyl phthalate, dipentyl phthalate, diisopentyl phthalate, n-pentyl isopentyl phthalate, dihexyl phthalate, bis(2-methoxyethyl) phthalate, and mixtures thereof.

22. The process as claimed in claim 1, wherein step b), and optionally steps f₁) and/or f₂), are performed at a temperature of between room temperature and 200° C., preferably between 40° C. and 180° C., at a pressure of between atmospheric pressure and 11.0 MPa, preferably between atmospheric pressure and 5.0 MPa, and for a time of between 1 minute and 10 hours, preferably between 10 minutes and 4 hours.

23. The process as claimed in claim 1, wherein step a) and/or step b), and optionally steps f₁) and/or f₂), are performed such that the mole ratio between the amount of said solvent (9) and the amount of said phthalate to be extracted or to be transformed is between 2 and 250, preferably between 4 and 90.

24. A process for recycling a PVC-based object containing at least one phthalate, including:
the conditioning of said PVC-based object comprising at least milling or shredding of said PVC-based object to form a PVC feedstock in the form of particles;
the recovery of a dialkyl phthalate and of a reusable target PVC plastic from said PVC feedstock in the form of particles as claimed in claim 1.

25. A process comprising manufacturing a flexible PVC-based object including a recycled PVC plastic and/or a dialkyl phthalate which are obtained via the process as claimed in claim 1.
